(19) United States
(12) Patent Application Publication
(10) Pub. No.: US 2024/0185967 A1
(43) Pub. Date: Jun. 6, 2024

(54) PATIENT-CUSTOMIZED ELECTRONIC MEDICAL QUESTIONNAIRE SYSTEM, MEDICAL QUESTIONNAIRE TERMINAL, AND MEDICAL QUESTIONNAIRE METHOD USING SAME

(71) Applicant: HD JUNCTION, INC., Seoul (KR)

(72) Inventors: Dong Hwan KIM, Seoul (KR); Seong Hun JIN, Seoul (KR)

(21) Appl. No.: 18/552,531

(22) PCT Filed: Nov. 22, 2021
(86) PCT No.: PCT/KR2021/017174

§ 371 Date: Sep. 26, 2023

(30) Foreign Application Priority Data
Apr. 22, 2021 (KR) 10-2021-0052279

(51) Int. Cl. G16H 10/20; G16H 10/60

(52) U.S. Cl. CPC G16H 10/20; G16H 10/60

(57) ABSTRACT

The present invention provides a patient-customized electronic medical questionnaire system, a medical questionnaire terminal using same, and a medical questionnaire method, the patient-customized electronic medical questionnaire system comprising: a medical questionnaire item generation unit for generating medical questionnaire items; an auxiliary answer generation unit for generating auxiliary answers for proposing to a patient with respect to the medical questionnaire items on the basis of medical record information about the patient; a communication interface for transmitting the medical questionnaire items and the auxiliary answers to the medical questionnaire terminal, and receiving, from the medical questionnaire terminal, answer information input by the patient; and a medical questionnaire result storage unit in which the answer information from the patient is stored.

### CROSS-REFERENCE TO RELATED APPLICATIONS

This application is the National Stage filing under 35 U.S.C. 371 of International Application No. PCT/KR2021/017174 filed on Nov. 22, 2021, which claims the benefit of Korean Patent Application No. 10-2021-0052279 filed on Apr. 22, 2021 the contents of which are all hereby incorporated by reference herein in their entirety.

### TECHNICAL FIELD

The present invention relates to a patient-customized electronic medical questionnaire system, a medical questionnaire terminal, and a medical questionnaire method using the same, and more particularly, to an electronic medical questionnaire system, a medical questionnaire terminal, and a medical questionnaire method using the same capable of improving convenience for patients and reliability of answers by using patient medical record information stored in a database.

### BACKGROUND ART

A medical questionnaire is performed to ascertain a condition of a patient in advance prior to performing medical activities such as medical treatment and examination, and a medical person performs medical activities in consideration of patient medical questionnaire information. A medical questionnaire procedure of the related art has been performed in a manner in which a patient manually writes an answer in a written medical questionnaire form, but recently, an electronic medical questionnaire scheme using a medical questionnaire terminal configured of an electronic device has been gradually expanded.

With the development of medical services, the number of medical questionnaire items is increased and the content of medical questionnaire are detailed in the medical questionnaire procedure so as to provide customized medical services based of patient information. However, this medical questionnaire procedure is cumbersome and difficult to answer from the point of view of the patient, and the reliability of patient answers tends to decrease as the content of the medical questionnaire is detailed.

### DISCLOSURE

### Technical Problem

The present invention is intended to solve the above problems, and to an electronic medical questionnaire system, a medical questionnaire terminal, and a medical questionnaire method using the same capable of improving convenience for patients and reliability of answers by using patient medical record information that can be accessed by a medical institution.

### Technical Solution

In order to achieve the above object, the present invention provides a patient-customized electronic medical questionnaire system including: a medical questionnaire item generation unit configured to generate a medical questionnaire item; an auxiliary answer generation unit configured to generate an auxiliary answer to be suggested to a patient for the medical questionnaire item on the basis of patient medical record information; a communication interface configured to transmit the medical questionnaire item and the auxiliary answer to a medical questionnaire terminal and receive answer information input by the patient from the medical questionnaire terminal; and a medical questionnaire result storage unit configured to store the answer information of the patient.

Here, when the answer information input for the medical questionnaire item by the patient does not match the auxiliary answer generated by the auxiliary answer generation unit, the medical questionnaire item generation unit may generate an additional medical questionnaire item based on the answer information input by the patient so that an additional medical questionnaire is able to be performed on the patient.

Meanwhile, the patient-customized electronic medical questionnaire system further includes: a reference information generation unit configured to generate reference information displayed to the patient through a medical questionnaire terminal so that the patient is able to refer to the reference information while creating a medical questionnaire, wherein the reference information generation unit generates the reference information on the basis of information associated with the medical questionnaire item in the patient medical record information.

A medical questionnaire result stored in the medical questionnaire result storage unit includes answer information of the patient for the medical questionnaire item and the auxiliary answer generated by the auxiliary answer generation unit.

Meanwhile, the object of the present invention described above can be achieved by a medical questionnaire terminal device including: a display unit configured to display a medical questionnaire item and an input cell to which a patient inputs an answer to the medical questionnaire item; and a communication interface configured to receive the medical questionnaire item from the medical questionnaire server and transmit an answer result input by the patient, wherein the display unit further displays an auxiliary answer to be suggested to the patient for the medical questionnaire item on the basis of patient medical record information.

Here, the display unit may further display a selection portion allowing the patient to select and input the same answer as the displayed auxiliary answer for the medical questionnaire item.

Further, the display unit may further display reference information that the patient is able to refer to for the medical questionnaire item while creating the medical questionnaire, and the reference information may be generated on the basis of information associated with the medical questionnaire item in the patient medical record information.

Meanwhile, the above object of the present invention can be achieved by a medical questionnaire method using a medical questionnaire terminal, including: generating a medical questionnaire item for a patient on the basis of information stored in a medical questionnaire item storage unit; generating an auxiliary answer to be suggested to the patient for the generated medical questionnaire item, on the basis of patient medical record information stored in a database; transmitting the medical questionnaire item and the auxiliary answer to the medical questionnaire terminal and displaying the medical questionnaire item and the auxiliary answer to the patient; and receiving an answer result input through the medical questionnaire terminal by the patient and storing the answer result in a medical questionnaire result storage unit, and can be achieved by a program stored in a medium or being downloadable as a program for performing the same.

Here, the medical questionnaire method further includes: generating reference information on the basis of information associated with the generated medical questionnaire item in the patient medical record information stored in the database, wherein the generated reference information is transmitted to the medical questionnaire terminal and displayed together with the medical questionnaire item.

The medical questionnaire method further includes: displaying an additional medical questionnaire item based on the answer information input through the medical questionnaire terminal by the patient when the answer information input by the patient does not match the auxiliary answer.

### Advantageous Effects

In the case of the present invention, since the medical questionnaire item is generated in consideration of characteristics of the patient, and the auxiliary answers and the reference information are provided on the basis of the patient medical record information, there is an advantage in that a patient-customized medical questionnaire is possible and the reliability of a medical questionnaire result is improved.

Further, since an answer can be made using the auxiliary answer and the reference information displayed through the medical questionnaire terminal, the answer convenience for the patient is improved.

Furthermore, there is an advantage in that it is possible to efficiently manage patient information by reflecting new patient information acquired through a patient medical questionnaire procedure in the patient medical record information.

### DESCRIPTION OF DRAWINGS

FIG. 1 is a diagram illustrating a main configuration of an electronic medical questionnaire system according to an embodiment of the present invention.

FIG. 2 is a detailed block diagram illustrating the main configuration of the medical questionnaire server of FIG. 1.

FIG. 3 is a diagram illustrating an example of a display unit of a medical questionnaire terminal.

FIG. 4 is a diagram illustrating another example of the display unit of the medical questionnaire terminal.

FIG. 5 is a diagram illustrating an example in which a patient inputs an answer in FIG. 4.

FIG. 6 is a diagram illustrating a data structure of medical questionnaire result data.

FIG. 7 is a flowchart illustrating a method of operating the electronic medical questionnaire system of FIG. 1 and a medical questionnaire method using the same.

### MODE FOR DISCLOSURE

Hereinafter, an electronic medical questionnaire system, a medical questionnaire terminal, and a medical questionnaire method using the same according to an embodiment of the present invention will be described in detail with reference to the drawings. In the following description, a positional relationship between respective components will be described with reference to drawings in principle. In the drawings, a structure of the invention may be simplified or exaggerated when necessary for convenience of description. However, the present invention is not limited thereto, and it is obvious that the present invention can be implemented by adding, changing, or omitting various devices.

Hereinafter, the ‘medical questionnaire’ refers to a procedure of inquiring about and confirming information on a state such as health of person. The medical questionnaire is not limited to a medical questionnaire performed for the purpose of medical treatment for patients by a medical institution, and includes a procedure in which various entities other than medical institutions inquire about and confirm personal condition information for various purposes, as in health examination or insurance subscription.

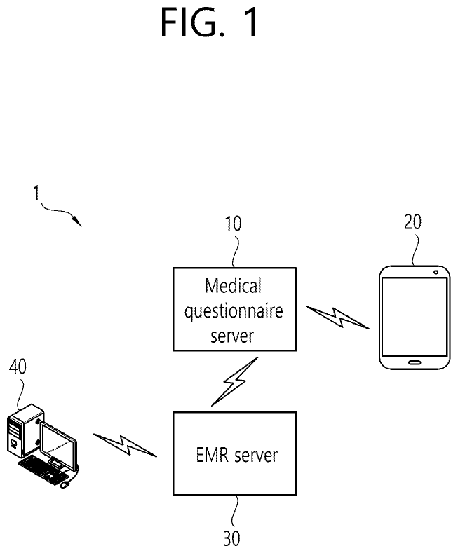

Hereinafter, an electronic medical questionnaire system according to an embodiment of the present invention will be described in detail with reference to FIG. 1.

FIG. 1 is a diagram illustrating a main configuration of an electronic medical questionnaire system 1 according to an embodiment of the present invention. The electronic medical questionnaire system 1 includes a medical questionnaire terminal 20 and a medical questionnaire server 10, and may further include an electronic medical record (EMR) server 30 and a medical staff terminal 40.

The medical questionnaire terminal 20 is a device that is carried by a patient and performs a medical questionnaire procedure. The medical questionnaire terminal 20 displays medical questionnaire items to the patient, and the patient inputs answer information for each medical questionnaire item through the medical questionnaire terminal. The medical questionnaire terminal 20 is configured of an electronic device in which a medical questionnaire program can be installed, and various electronic devices having a display element such as a PC, a netbook, a tablet PC, and a smart phone are used. The medical questionnaire terminal may be a terminal provided by a medical institution. However, the medical questionnaire terminal is not limited thereto, and a necessary program may be installed in a terminal owned by a user and the terminal may be used.

The medical questionnaire server 10 is configured to generate medical questionnaire items for a medical questionnaire of a patient and store answer information input through the medical questionnaire terminal by the patient. The medical questionnaire server 10 is communicatively connected to devices such as the medical questionnaire terminal 20, the electronic medical record server 30, and the medical staff terminal 40, and transmits or receives information necessary for the medical questionnaire procedure. The medical questionnaire server 10 includes a memory for storing various pieces of data and a processor for computing and processing necessary information. For the medical questionnaire server 10, a server installed in a medical institution may be used or a cloud server capable of computing and storage in a cloud may be used. Although the medical questionnaire server is configured as a separate configuration in FIG. 1, the medical questionnaire server may be configured using a processor and a memory included in the medical questionnaire terminal or the medical staff terminal, instead of being configured as the separate server. A specific configuration of the medical questionnaire server will be separately described hereinafter.

The electronic medical record server 30 is configured to store the medical record information of the patient. The electronic medical record server 30 includes a storage device such as a memory, and accumulates various types of health and medical information from the past to the present, such as patient personal information, hospital visit information, diagnosis information, prescription information, and examination information. The electronic medical record server 30 is configured to be communicatively connected to devices such as the medical questionnaire server 10 and the medical staff terminal 40. Accordingly, the electronic medical record server 30 receives and stores the patient medical record information input from the medical staff terminal, or provides the patient medical record information upon request from the medical staff terminal. Further, the electronic medical record server 30 may provide the patient medical record information requested by the medical questionnaire server, and may receive patient medical questionnaire results and update the patient medical record information. For the electronic medical record server 30, a server installed in a medical institution or a cloud server may be used, as in the medical questionnaire server described above. Although the medical questionnaire server and the electronic medical record server are illustrated as separate configurations in FIG. 1, the present invention is not limited thereto, and the servers may be configured as a single server for performing both functions of the examination server and functions of the electronic medical record server.

The medical staff terminal 40 is configured of an electronic device having a display element like the medical questionnaire terminal, and is communicatively connected to a device such as the electronic medical record server 30 and the medical questionnaire server 10. The medical staff may view the patient medical record information through the medical staff terminal 40 and transmit a necessary request to another device (for example, a request for a medical questionnaire of a specific patient to the medical questionnaire server).

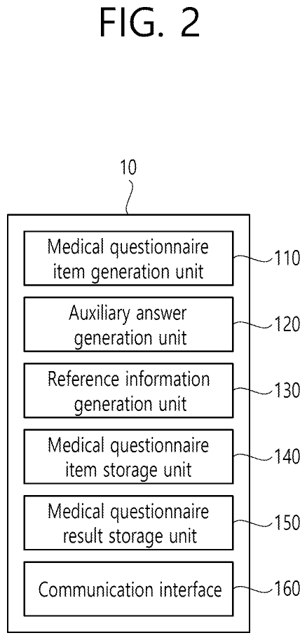

FIG. 2 is a detailed block diagram illustrating a main configuration of the medical questionnaire server of FIG. 1. Hereinafter, the configuration of the medical questionnaire server will be described in more detail with reference to FIG. 2.

As described above, the medical questionnaire server 10 is configured to generate medical questionnaire items for a patient medical questionnaire procedure and store medical questionnaire results as an answer to each medical questionnaire item input by the patient. Furthermore, the medical questionnaire server of the present embodiment may generate various types of content that the patient may utilize and refer to in an answering process at the time of the medical questionnaire, in addition to the medical questionnaire items.

As illustrated in FIG. 2, the medical questionnaire server 10 according to the embodiment includes a medical questionnaire item generation unit 110, an auxiliary answer generation unit 120, a reference information generation unit 130, a medical questionnaire item storage unit 140, a medical questionnaire result storage unit 150, and a communication interface 160. Here, each of the medical questionnaire item generation unit, the auxiliary answer generation unit, and the reference information generation unit may include a separate processor and memory, or may be configured using an integrated processor and/or integrated memory.

First, the communication interface 160 includes a communication module, and configured to perform communicative connection so that various types of information can be transmitted or received between the medical questionnaire server 10 and an external device (for example, a medical questionnaire terminal, an electronic medical information server, a medical staff terminal, or a patient device such as a personal healthcare device). The communication interface 160 can receive or transmit necessary data from or to the external device described above via a communication network using various known wired or wireless communication schemes.

The medical questionnaire item generation unit 110 generates medical questionnaire items used in the medical questionnaire procedure. Here, the medical questionnaire items may be inquiry items or request items for checking patient health information. The medical questionnaire item generation unit 110 generates a plurality of medical questionnaire items for obtaining patient information using the information stored in the medical questionnaire item storage unit 140.

The medical questionnaire item generation unit 110 may generate patternized medical questionnaire items according to a preset policy. For example, the medical questionnaire item generation unit 110 may be configured to generate pre-set medical questionnaire items according to the purpose of the medical questionnaire, such as a medical questionnaire for young children, an allergy medical questionnaire, and a prior medical questionnaire for vaccination.

Alternatively, the medical questionnaire item generation unit 110 may also generate patient-customized medical questionnaire items on the basis of the patient medical record information rather than the patternized medical questionnaire items. In the case of the patternized medical questionnaire items, since characteristics of patients are not considered, unnecessary questions are included or there is a limit to obtaining detailed information considering the characteristics of the patient. Accordingly, the medical questionnaire server 10 of the present embodiment receives patient medical record information from the electronic medical record server, and the medical questionnaire item generation unit 110 generates patient-customized medical questionnaire items on the basis of the patient medical record information. Specifically, a sex, age, medical treatment record, examination record, prescription information, previous medical questionnaire result, and the like of the patient may be considered when the medical questionnaire items are generated, and furthermore, a medical staff request made through the medical staff terminal may also be considered.

As mainly described here, the patient medical record information is provided from the electronic medical record server, but this is an example and the patient medical record information may be acquired in various ways. For example, the patient medical record information may be previous patient medical questionnaire results stored in the medical questionnaire server itself, may be various types of information (monitoring information, measurement information, input information, or the like) transmitted by a personal healthcare device of the patient, or may be patient examination information stored in an examination device or the like, or patient information stored in a server of the Health Insurance Review and Assessment Service or an insurance company. Therefore, although a scheme of transmitting the patient medical record information from the electronic medical record server is adopted an example in describing the patient medical record information, this should be construed with extension to the patient medical record information provided from various external devices other than the electronic medical record server.

The medical questionnaire item storage unit 140 includes a medical questionnaire item database for generating medical questionnaire items, and is configured to include a storage device such as a memory. In the database, a plurality of medical questionnaire item samples to be used in the medical questionnaire procedure are stored, and the medical questionnaire item samples are classified depending on the purpose of medical questionnaire and other preset criteria and stored.

As an example, a set of medical questionnaire items for each purpose of medical questionnaire may be stored in the database. The medical questionnaire item set is a group of a plurality of medical questionnaire item samples required for the purpose, and some of the medical questionnaire item samples included in one of the sets form a hierarchical relationship. Accordingly, when a medical questionnaire request is received from the medical staff terminal, the medical questionnaire item generation unit 110 may select a necessary sample depending on a patient medical record in the set according to a requested medical questionnaire purpose to generate a medical questionnaire item.

Each medical questionnaire item sample may be stored as detachable module type data. For example, a medical questionnaire item sample configured in the form of a sentence is configured such that a subject, an object, and a predicate are separately edited as module-type data. Accordingly, the medical questionnaire item generation unit 110 may generate patient-customized medical questionnaire items by editing some data modules in samples in consideration of the patient medical record information at the time of generating the medical questionnaire items.

Meanwhile, the medical questionnaire server 10 of the present embodiment further includes an auxiliary answer generation unit 120 and a reference information generation unit 130. The auxiliary answer generation unit 120 and the reference information generation unit 130 are configured to generate an auxiliary answer and reference information for each medical questionnaire item, respectively, and the auxiliary answer and the reference information may be generated by using the patient medical record information stored in the electronic medical record server 30 described above.

Specifically, when a medical questionnaire item is created in the medical questionnaire item generation unit 110, the auxiliary answer generation unit 120 generates auxiliary answers to be suggested to the patient for each medical questionnaire item. These auxiliary answers are preliminary answers estimated for each medical questionnaire item by the server itself, and are generated by being estimated on the basis of information related to the medical questionnaire item in the patient medical record (including previously created medical questionnaire result information) stored in the electronic medical record server or the like.

As an example, the database of the medical questionnaire item storage unit 140 defines a scheme of estimating an auxiliary answer for each medical questionnaire item sample, and the auxiliary answer generation unit generates an auxiliary answer on the basis of the defined scheme from the patient medical record information. For example, for a medical questionnaire about whether or not there are hepatitis antibodies, a definition may be made so that an auxiliary answer is estimated on the basis of a previous examination record of the patient. Alternatively, for a medical questionnaire item for confirming whether or not to takea drug for a chronic disease, a definition may be made so that an auxiliary answer is estimated on the basis of previous prescription information of the patient. However, the auxiliary answers are not generated for all the medical questionnaire items. An auxiliary answer estimation scheme is defined only for medical questionnaire items of which answers can be estimated through a past patient medical record, and auxiliary answers are not generated for medical questionnaire items for which the estimation scheme is not defined. Further, even when the estimation scheme is defined, an auxiliary answer may not be generated in a case in which the patient medical record information does not include information on items necessary for estimation.

Meanwhile, the reference information generation unit 130 generates reference information to be provided to the patient for each medical questionnaire item, as described above. The reference information is information that the patient can refer to when determining the answer of the patient to each medical questionnaire item. The reference information may be generated from information related to the medical questionnaire item in the patient medical record. As an example, the database of the medical questionnaire item storage unit 140 defines a scheme for generating the reference information for each medical questionnaire item sample, and the reference information generation unit 130 generate the reference information on the basis of the defined scheme from the patient medical record information. For example, when the medical questionnaire item is a medical questionnaire item for confirming whether or not a vaccination is received, a vaccination record may be provided as the reference information on the basis of patient prescription information, and when the medical questionnaire item is a medical questionnaire item for asking whether an anticancer drug or radiation treatment has been recently received, a recent anticancer drug prescription record and a radiotherapy record may be provided as the reference information.

Such reference information can also be generated using other information related to the medical questionnaire items (for example, description of terms included in the medical questionnaire items), as well as being generated on the basis of the patient medical record information. Further, the reference information may be generated for some medical questionnaire items, not all the medical questionnaire items, like the auxiliary answer described above.

The auxiliary answer and the reference information generated by the above-described scheme may be transmitted to the medical questionnaire terminal 20 and displayed to the patient. Since the patient decides an answer to each medical questionnaire item in consideration of the auxiliary answers and the reference information suggested through the terminal during the medical questionnaire procedure, the convenience of the answer by the patient is improved and a highly reliable answer result can be obtained.

The result of the answer of the patient performed through the medical questionnaire terminal 20 is transmitted to the medical questionnaire server through the communication interface 160, and the transmitted answer information is stored in the medical questionnaire result storage unit 150. The medical questionnaire result storage unit 150 includes a storage device such as a memory, and the stored answer results are transmitted to the electronic medical record server 30 through the communication interface to update the patient medical record information or is transmitted to the medical staff terminal so that the answer results can be displayed to a medical staff. Detailed description of the medical questionnaire result storage unit will be described in detail below with reference to separate drawings.

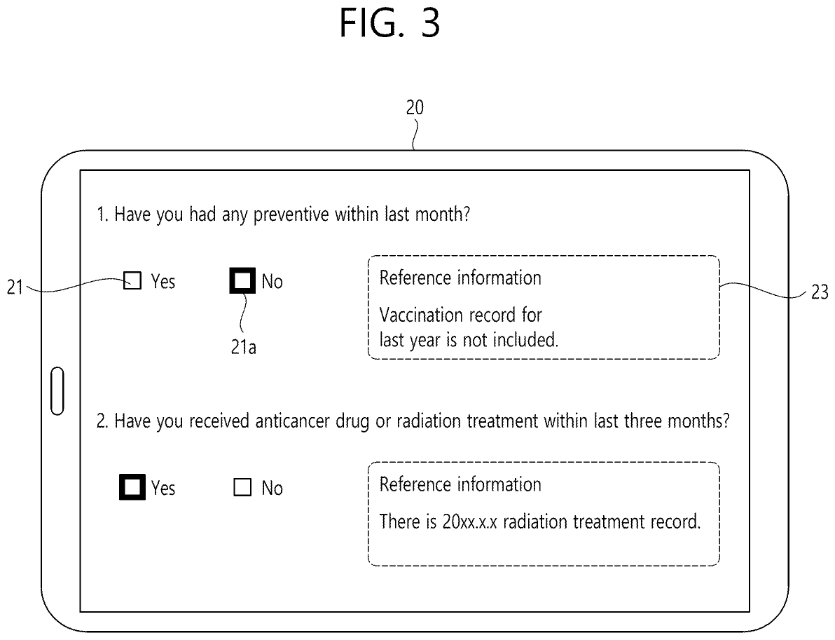

FIGS. 3 to 5 are diagrams illustrating examples of a display unit of the medical questionnaire terminal. Hereinafter, the medical questionnaire terminal and various contents displayed through the terminal during the medical questionnaire procedure will be described in detail with reference to FIGS. 3 to 5.

As described above, the medical questionnaire terminal 20 is configured of an electronic device and displays various medical questionnaire contents to the patient, and the patient inputs an answer through the medical questionnaire terminal. The medical questionnaire terminal 20 includes a communication interface, a processor, a memory, and a display unit.

The communication interface is configured of a communication module capable of transmitting or receiving data to or from a communication interface of the medical questionnaire server. Using this, the medical questionnaire terminal receives information such as medical questionnaire items, the auxiliary answer, and the reference information generated by the medical questionnaire server, and transmits an answer result input by the patient to the medical questionnaire server.

A program for patient medical questionnaire is installed in the memory, and the processor performs computing work and processing work according to instructions of the installed program. Data such as the medical questionnaire items, the auxiliary answers, and the reference information transmitted from the medical questionnaire server is stored in the memory, and the processor generates necessary content using the stored data and displays the content through the display unit.

FIG. 3 is a diagram illustrating an example of the display unit of a medical questionnaire terminal. The display unit is configured of a display element such as a display, and generated medical questionnaire items and relevant answer items in FIG. 3 are displayed as illustrated in FIG. 3.

The medical questionnaire items displayed on the display unit are the medical questionnaire items generated by the medical questionnaire item generation unit 110 described above, and when the medical questionnaire item is configured of a selective type question, selectable answer items are displayed together. Further, an input cell 21 to which the patient inputs an answer for each medical questionnaire item is displayed on the display unit, and a user inputs an answer to the input cell using an input device such as a mouse, keyboard, or digitizer. An answer scheme is variously configured according to the medical questionnaire items (for example, a choice type or a short answer type), and the input cell may be variously configured as a choice type, a short answer type, or the like according to the answer scheme of the medical questionnaire item.

Meanwhile, the auxiliary answer information generated by the auxiliary answer generation unit 120 described above is also displayed to the patient through the display unit. The auxiliary answer is displayed in the input cell or a position adjacent to the input cell, and may be displayed in various schemes according to an answer scheme of the input cell. However, the auxiliary answer is displayed in an auxiliary and preliminary manner so that the patient can select another answer without following the auxiliary answer at the time of medical questionnaire.

Further, a reference information display area 23 in which the reference information for each medical questionnaire item is displayed is formed on the display unit, and the reference information generated by the reference area generation unit described above is displayed. Here, the reference information may be information related to each medical questionnaire item in the patient medical record information, and may be information that is a basis for estimating the auxiliary answer. The reference information is displayed separately for each medical questionnaire item, and is disposed at a position adjacent to the answer item of each medical questionnaire.

As an example, referring to the content displayed in FIG. 3, the medical questionnaire items generated by the medical questionnaire item generation unit 110 and displayed on the display unit are questions for confirming whether or not vaccination has been recently received. The reference information generation unit 130 checks a vaccination record in the patient medical record information and generates information indicating that there is no vaccination record for the last year as the reference information. The generated reference information is displayed in the reference information display area 23, as illustrated in FIG. 3. The auxiliary answer generation unit 120 generates an auxiliary answer of ‘no’ in consideration of the fact that there is no recent vaccination record in the patient medical record, and this auxiliary answer 21a is displayed on the display unit. As illustrated in FIG. 3, the auxiliary answer 21a is displayed in a way that the input cell of the answer item is displayed in bold. However, the auxiliary answer display way is not limited thereto, and may be displayed in various ways by displaying a separate mark or using shade, color, or the like. When the content of the medical questionnaire including the medical questionnaire items, auxiliary answer, and reference information as described above are displayed through the display unit, the patient considers the reference information for the medical questionnaire item and inputs an answer by referring to the suggested auxiliary answer.

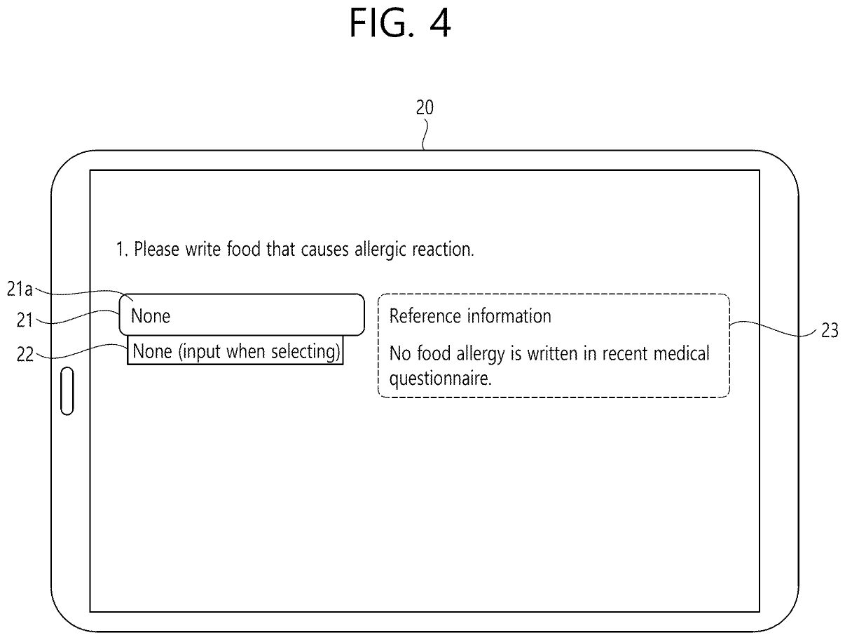

FIG. 4 is a diagram illustrating another example of the display unit of the medical questionnaire terminal. As illustrated in FIG. 4, the medical questionnaire item is a question for asking food allergy information. According to the patient medical record information, there is a record in which a patient has answered that the patient has not have a separate allergy in a recent medical questionnaire, content thereof is displayed as the reference information, and the absence of food allergy is displayed as the auxiliary answer from the reference information.

Here, an answer scheme for the medical questionnaire items is not a selective answer scheme as illustrated in FIG. 3, but a scheme for inputting a short answer to the input cell. In this case, the auxiliary answer 21a for the medical questionnaire item may be displayed inside the input cell 21. Although not clearly identified on the drawing, the auxiliary answer 21a may be displayed using a different color such as gray or with different transparency so that the auxiliary answer can be displayed separately from an answer directly input by the patient. Thereafter, the patient determines an answer in consideration of the auxiliary answer, and inputs the content of the answer to the input cell 21. In this case, the auxiliary answer displayed inside the input cell may be deleted during an input operation by the patient in order to prevent confusion at the time of input.

When the patient wants to answer with the same content as the auxiliary answer, the patient can also conveniently input the short answer type answer by selecting the auxiliary answer through a selection portion 22 without directly inputting the short answer type answer. As illustrated in FIG. 4, the patient may select the selection portion displayed below the input cell 21 and answer with the same content as the auxiliary answer at the time of an input operation.

An example of a display scheme of the auxiliary answer and the selection portion is illustrated in FIG. 4, but the present invention is not limited thereto, and various other ways may be used. For example, the auxiliary answer can be configured to be displayed at a position adjacent to the input cell instead of the inside of the input cell, and the selection portion can allow the same answer to be selected by checking a separate cell instead of a window form.

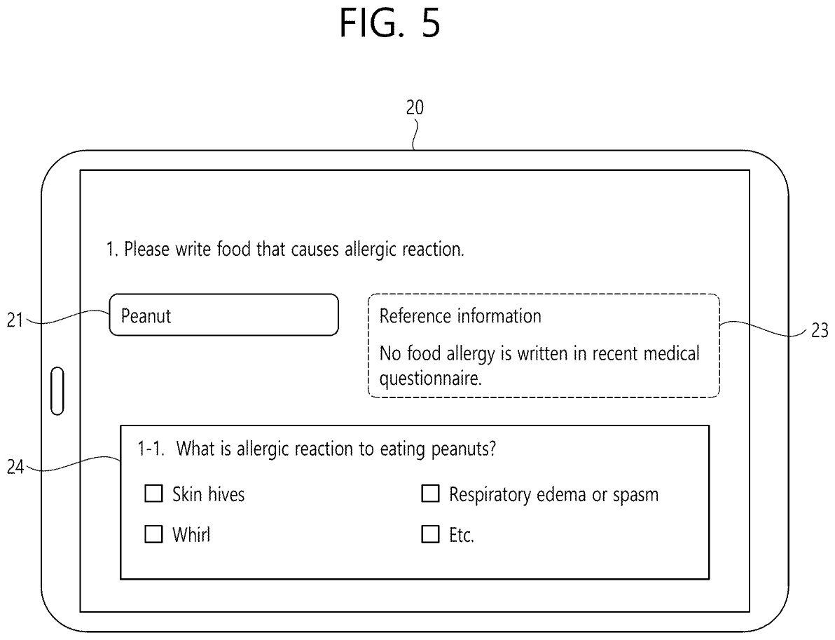

FIG. 5 is a diagram illustrating an example in which the patient inputs the answer in FIG. 4. As described in FIG. 4, ‘none’ has been suggested as the auxiliary answer to the medical questionnaire item, but FIG. 5 illustrates a case in which the patient inputs ‘peanuts’ as an answer, unlike the auxiliary answer. Thus, when the auxiliary answer and an answer result of the patient are different, the medical questionnaire terminal 20 may perform an additional medical questionnaire by displaying additional medical questionnaire items according to the answer result of the patient. In this case, the additional medical questionnaire item may be displayed on an additional medical questionnaire window 24, and the additional medical questionnaire window 24 may be selectively displayed on the display unit when an additional medical questionnaire condition occurs.

More specifically, the medical questionnaire item generation unit 110 is configured to generate the plurality of necessary medical questionnaire items in consideration of the purpose of the medical questionnaire and the patient medical record information, in which the plurality of medical questionnaire items have a hierarchical relationship. Here, not all generated medical questionnaire items are displayed on the display unit, but a definition can be made so that medical questionnaire items corresponding to a default value are displayed, and the other medical questionnaire items are selectively displayed according to a specific condition. When the answer of the patient to the medical questionnaire item satisfies a preset condition, it is possible to selectively display medical questionnaire items according to the condition. Further, when the answer of the patient to the medical questionnaire item is different from the suggested auxiliary answer, it is also possible to selectively display the medical questionnaire items according to the condition.

When all the medical questionnaire items (that is, all the medical questionnaire items considering all conditions) generated by the medical questionnaire item generation unit 110 are displayed through the display unit, too many unnecessary medical questionnaire items are included and concentration of answers may be reduced. On the other hand, according to the present embodiment, it is possible to perform the medical questionnaire with only medical questionnaire items necessary for the patient, other than the unnecessary medical questionnaire items. Further, when an additional medical questionnaire is performed according to answer conditions for the patient, it is possible to acquire detailed information of the patient related to the answer. Furthermore, it is possible to confirm a reason for mismatch that could not be confirmed from existing patient medical record information and update the patient medical record information with the reason, by performing an additional medical questionnaire on condition that the auxiliary answer does not match the answer of the patient (for example, when the patient has answered that the patient had recently received the vaccination unlike the auxiliary answer in FIG. 3, it is possible to confirm what type of vaccination has been received in a certain way through an additional medical questionnaire item).

When the answer input of the patient is completed through the scheme described above, the answer information of the patient is stored in the memory of the medical questionnaire terminal 20, and then stored in the medical questionnaire result storage unit 150 of the medical questionnaire server through the communication interfaces of the terminal and the server.

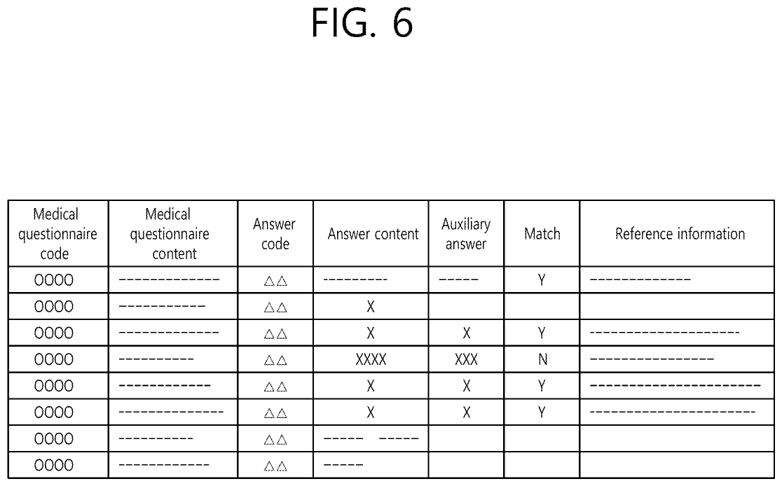

FIG. 6 is a diagram illustrating a data structure of medical questionnaire result data. The medical questionnaire result data stored in the medical questionnaire result storage unit 150 is stored in a form in which each medical questionnaire item is associated with the answer result of the patient to the medical questionnaire item. In addition, auxiliary answers and reference information for the medical questionnaire items may also be stored in an associated form. Furthermore, whether the answer result of the patient matches the auxiliary answer of the patient may also be stored in an associated form.

The medical questionnaire result data stored in this way is transmitted to the electronic medical record server 30 and is cumulatively stored and updated in the patient medical record information or transmitted to the medical staff terminal 40 so that the medical staff can view and utilize the medical questionnaire result data for medical treatment. In particular, in the case of a data format according to FIG. 6, when the auxiliary answer based on existing patient medical record information does not match the answer result of the patient, information related to the medical questionnaire item in the existing patient medical record information may be updated or the existing information can be modified and managed. Further, when the medical questionnaire result or the patient medical record information is displayed on the medical staff terminal 40, information related to the medical questionnaire item for which the auxiliary answer and the answer result do not match each other is highlighted, so that the medical staff can easily identify change in condition of the patient and recent information and perform medical treatment.

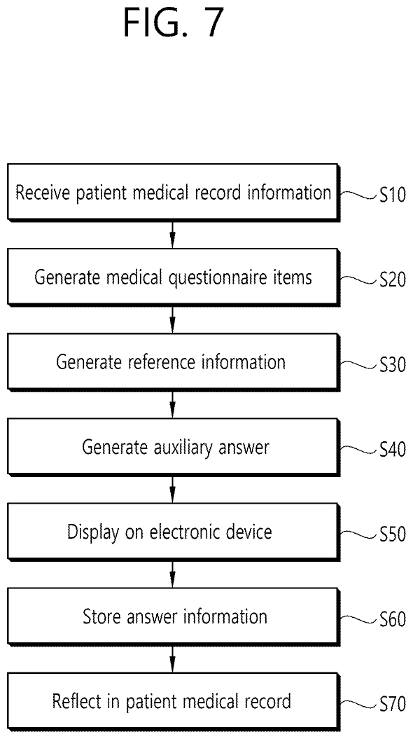

FIG. 7 is a flowchart illustrating a method of operating an electronic medical questionnaire system of FIG. 1 and a medical questionnaire method using the same. Hereinafter, a method for operating an electronic medical questionnaire system according to the embodiment and a medical questionnaire method using the same will be described in detail with reference to FIG. 7.

First, when a medical questionnaire order is received from the medical staff terminal 40 or a separate reception terminal to the electronic medical questionnaire system, the medical questionnaire server 10 receives the patient medical record to generate customized medical questionnaire content for the patient (S10). Here, for the patient medical record, information stored in the electronic medical record server 30 can be also received, or the patient medical record can also be received from a personal healthcare device, an examination device, a server of the Health Insurance Review and Assessment Service or an insurance company, or the like.

The medical questionnaire item generation unit 110 of the medical questionnaire server generates the medical questionnaire items in consideration of the purpose of the medical questionnaire included in the medical questionnaire order, a request of the medical staff, and the like, and furthermore, generates customized medical questionnaire items in consideration of the pre-stored patient medical record information. (S20). In this case, the generated medical questionnaire item may be generated on the basis of the medical questionnaire item sample stored in the medical questionnaire item storage unit by editing the sample to reflect patient information. Here, the plurality of medical questionnaire items form a hierarchical relationship, and some of the medical questionnaire items may be defined to be conditionally displayed on the display unit as described above.

When the plurality of medical questionnaire items are generated, the reference information generation unit 130 generates reference information for each medical questionnaire item (S30). In this case, the reference information may be generated using information related to the medical questionnaire item in the received medical record information of the patient.

In addition, the auxiliary answer generation unit 120 generates the auxiliary answer information for each medical questionnaire item (S40). The auxiliary answer may be an answer estimated from information related to the medical questionnaire item in the received patient medical record information.

In FIG. 7, it is shown that the medical questionnaire item, the reference information, and the auxiliary answer are generated using the patient medical record information initially received in step S10, but the present invention is not limited to this order, and necessary patient medical record information may be requested, received, viewed, and used from time to time in respective steps from S20 to S40.

Medical questionnaire content (for example, the medical questionnaire items, reference information, auxiliary answers) generated through the above steps is transmitted from the medical questionnaire server 10 to the medical questionnaire terminal 20, and the medical questionnaire terminal 20 processes such information and displays the information through the display unit of the terminal (S50). In this case, the medical questionnaire is performed in such a way that the display unit displays the reference information and the auxiliary answers for each medical questionnaire item together, and the patient determines and inputs an answer in consideration of such information.

When the answer of the patient is completed, the medical questionnaire result information including the answer result of the patient is stored in the medical questionnaire terminal and stored in the medical questionnaire server through a communication interface (S60). Information collected through the medical questionnaire procedure is then updated and reflected in the patient medical record information stored in the electronic medical record server or the like (S70), and the medical staff can perform medical treatment by referring to the stored medical questionnaire results or the updated patient medical record information (the patient medical record information can be updated and reflected by reflecting the results of the medical questionnaire in an external device that has provided the patient medical record information, in addition to the electronic medical record server).

In the case of the electronic medical questionnaire system, medical questionnaire terminal, and medical questionnaire method described above, there are advantages that the convenience for the patient can be improved and reliable medical questionnaire results can be obtained since the patient performs the medical questionnaire in consideration of the reference information and the auxiliary answers.

The method of operating the medical questionnaire system and the medical questionnaire method according to the present embodiment described above can be achieved by a program (including an application) capable of performing such a process being installed and executed in the server or the medical questionnaire terminal. Accordingly, the present invention can be implemented by such a program (a program stored in a medium or being downloadable) or a medium having such a program stored therein.

Such a program may include code coded in a program language readable by a computing device of the electronic device so that the electronic device reads the program and executes the methods implemented as a program. Such code may include functional code related to, for example, functions defining necessary functions for executing the methods, and may include control code related to an execution procedure necessary for a computing device of the terminal to execute the functions according to a predetermined procedure.

Further, a medium in which such a program is stored is not a medium that stores data for a short moment, such as a register, cache, or memory, but is a medium that stores data semi-permanently and can be read by a device. Specifically, examples of the medium in which the program is stored include a ROM, a RAM, a CD-ROM, a magnetic tape, a floppy disk, and an optical data storage device, but the present invention is not limited thereto. For example, the program may be stored in various recording media on various web servers that can be accessed by electronic devices. Further, it should be noted that the medium may be distributed in computer systems connected by a network, and computer-readable code may be stored in a distributed manner.

The embodiment of the present invention has been described above in detail, but the present invention is not limited to the embodiment. It should be noted that those skilled in the art to which the present invention belongs can make various modifications or changes to the present invention without departing from the scope of the technical characteristics of the present invention defined in the appended claims.

1. A patient-customized electronic medical questionnaire system comprising:
a medical questionnaire item generation unit configured to generate a medical questionnaire item;
an auxiliary answer generation unit configured to generate an auxiliary answer to the medical questionnaire item on the basis of patient medical record information;
a communication interface configured to transmit the medical questionnaire item and the auxiliary answer to a medical questionnaire terminal and receive answer information input by a patient from the medical questionnaire terminal; and
a medical questionnaire result storage unit configured to store the answer information of the patient.

2. The patient-customized electronic medical questionnaire system of claim 1, wherein the medical questionnaire item generation unit generates the medical questionnaire item on the basis of the patient medical record or a request from a medical staff.

3. The patient-customized electronic medical questionnaire system of claim 1, wherein the patient medical record information includes at least one of patient medical record information stored in the electronic medical record server, previously performed medical questionnaire result information, and information received from a personal device of the patient.

4. The patient-customized electronic medical questionnaire system of claim 1, wherein, when the answer information input for the medical questionnaire item by the patient does not match the auxiliary answer generated by the auxiliary answer generation unit, the medical questionnaire item generation unit generates an additional medical questionnaire item based on the answer information input by the patient so that an additional medical questionnaire is able to be performed on the patient.

5. The patient-customized electronic medical questionnaire system of claim 1, further comprising:
a reference information generation unit configured to generate reference information displayed to the patient through a medical questionnaire terminal so that the patient is able to refer to the reference information while creating a medical questionnaire,
wherein the reference information generation unit generates the reference information on the basis of information associated with the medical questionnaire item in the patient medical record information.

6. The patient-customized electronic medical questionnaire system of claim 1, wherein a medical questionnaire result stored in the medical questionnaire result storage unit includes answer information of the patient for the medical questionnaire item and the auxiliary answer generated by the auxiliary answer generation unit.

7. The patient-customized electronic medical questionnaire system of claim 1, wherein the medical questionnaire terminal includes an interface for displaying medical questionnaire items generated by the medical questionnaire item generation unit, and input cells to which a patient inputs answers to the medical questionnaire items, and
the interface displays the auxiliary answer generated by the auxiliary answer generation unit to a user.

8. A medical questionnaire terminal device comprising:
a display unit configured to display a medical questionnaire item and an input cell to which a patient inputs an answer to the medical questionnaire item; and
a communication interface configured to receive the medical questionnaire item from the medical questionnaire server and transmit an answer result input by the patient,
wherein the display unit further displays an auxiliary answer to be suggested to the patient for the medical questionnaire item on the basis of patient medical record information.

9. The medical questionnaire terminal device of claim 8, wherein the display unit further displays a selection portion allowing the patient to select and input the same answer as the displayed auxiliary answer for the medical questionnaire item.

10. The medical questionnaire terminal device of claim 8,
wherein the display unit further displays reference information that the patient is able to refer to for the medical questionnaire item while creating the medical questionnaire, and
the reference information is generated on the basis of information associated with the medical questionnaire item in the patient medical record information.

11. A medical questionnaire method using a medical questionnaire terminal, comprising:
generating a medical questionnaire item for a patient on the basis of information stored in a medical questionnaire item storage unit;
generating an auxiliary answer to be suggested to the patient for the generated medical questionnaire item, on the basis of patient medical record information stored in a database;
transmitting the medical questionnaire item and the auxiliary answer to the medical questionnaire terminal and displaying the medical questionnaire item and the auxiliary answer to the patient; and
receiving an answer result input through the medical questionnaire terminal by the patient and storing the answer result in a medical questionnaire result storage unit.

12. The medical questionnaire method using a medical questionnaire terminal of claim 11,
further comprising:
generating reference information on the basis of information associated with the generated medical questionnaire item in the patient medical record information stored in the database,
wherein the generated reference information is transmitted to the medical questionnaire terminal and displayed together with the medical questionnaire item.

13. The medical questionnaire method using a medical questionnaire terminal of claim 11, further comprising:
displaying an additional medical questionnaire item based on the answer information input through the medical questionnaire terminal by the patient when the answer information input by the patient does not match the auxiliary answer.

14. The medical questionnaire method using a medical questionnaire terminal of claim 11, wherein, in the storing of the medical questionnaire result, the medical questionnaire result includes the answer information of the patient and the auxiliary answer for the medical questionnaire item.

15. A computer program stored in a medium or being downloadable, for executing the method of claim 11.
